(19) United States
(12) Patent Application Publication
(10) Pub. No.: US 2010/0106145 A1
(43) Pub. Date: Apr. 29, 2010

(54) ABLATION TECHNIQUE FOR COSMETIC SURGERY

(76) Inventor: Alan David Widgerow, Sandton (ZA)

Correspondence Address: SMITH FROHWEIN TEMPEL GREENLEE BLAHA, LLC, Two Ravinia Drive, Suite 700, ATLANTA, GA, 30346, US

(21) Appl. No.: 12/531,858

(22) PCT Filed: Mar. 18, 2008
(86) PCT No.: PCT/ZA08/00020

§ 371 Date: Sep. 17, 2009

Related U.S. Application Data
(60) Provisional application No. 60/896,273, filed on Mar. 22, 2007.

(51) Int. Cl. A61B 18/20; A61B 18/04

(52) U.S. Cl. 606/12; 606/33

(57) ABSTRACT

A method of non-invasive treatment of the human body is described by ablating a muscle or a nerve controlling the muscle. The method comprises stimulating a muscle to emit an electromyographic signal. The electromyographic signal is detected to ascertain the location of the said muscle or alternatively the nerve. The muscle or nerve is heated by means of optical energy while cooling the skin in the vicinity of the said muscle or nerve. Then radio frequency energy is applied to the muscle or nerve to ablate or partially to ablate said muscle or nerve.

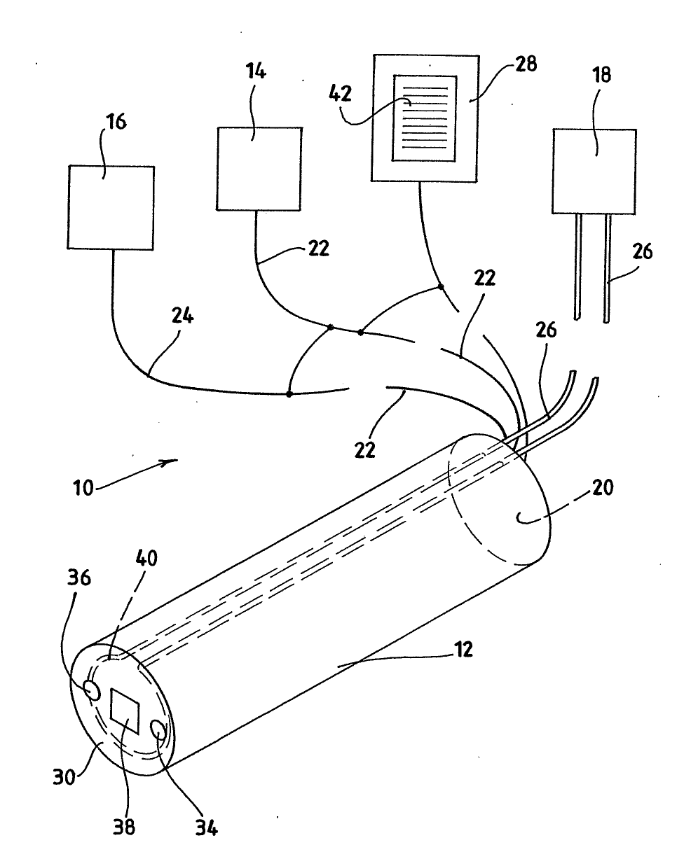

This invention relates to apparatus for use in methods of cosmetic surgery and to such methods.

Aging is associated with the development of lines and wrinkles caused by actinic damage, gravitational effect, sleep lines, and muscular action. Skin wrinkles are grooves in the skin. Wrinkles can be on the surface of the skin or can be quite deep. Wrinkles may be categorized as either static (persistent, permanent) or dynamic (expression related). Dynamic facial wrinkles occur due to movement or muscle activity in the affected area, associated with repeated facial movements, such as frowning or even heavy natural use of facial expressions (raising eyebrows, laughing, etc). Dynamic wrinkles (our focus) will improve by reducing the muscle activity in the affected area.

Many facial muscles of expression insert on to the undersurface of the skin. Following contraction, including baseline muscle tone, a visible line or wrinkle will develop in the skin that will deepen permanently over time. This type of wrinkle cannot be removed by any type or superficial skin treatment, but can only be treated at the source: the muscle itself.

Facial expression depends on the balance of agonist-antagonist muscle interactions. As an example the brow position is governed by a balance between elevator (frontalis) and depressor (orbicularis; corrugators; procerus) muscles. If the depressor muscles are weakened or paralysed the elevators become dominant and the brow position rises. The same is true for other areas. The corners of the lips are elevated (levator anguli oris) or depressed (depressor anguli oris) by different muscles, thus to lift the corner of the mouth depressors would be weakened or paralysed. This theory is adapted to many areas of the face.

Aside from altering the status of muscle balance, individual muscles may be selected for weakening or paralysis to diminish its affect on the skin in that particular area.

Known techniques for ablation of the nerves causing muscle movement are known. These include the use of toxins such as Botulinum toxin (known commercially as Botox® and Dysport®) which are produced by bacteria called Clostridium botulinum. Botox® acts by blocking acetylcholine, a chemical that is responsible for transmitting electrical impulses that cause muscle contraction. This results in muscle paralysis. The resultant paralysis, however, is temporary, as the new growth of nerves will re-innervate the muscles. This treatment has disadvantages making the process inefficient. Experience has shown that these are inaccurate in the their nerve targeting and unpredictable in their efficiency and the length of time of this effect.

Direct excision of nerves and electric ablation is very invasive and destructive. It involves side effects of scarring, substantial surrounding tissue damage and necessity for significant anaesthesia. Previous attempts at radio frequency (RF) ablation of the nerve group involved the use of a percutaneous insertion of an insulated bipolar needle vertically through the eyebrow skin to entrap the corrugator nerve plexus. This too is invasive and causes tissue damage as well as being rather inefficient.

U.S. Pat. No. 7,238,183 (Kreindel) describes a method for treating a skin target being a skin defect such as a vascular lesion, pigmented lesion, acne, unwanted hair or wrinkle. The method includes heating the target by optical means, cooling the skin and then applying radio frequency heating to the target. This will affect the target e.g. by ablating muscle. Although this process is a significant step forward because it is non-invasive, the process suffers from the disadvantage that, especially when working with wrinkles in the face, the location of the target being treated transcutaneously cannot be accurately determined by the surgeon. As there are so many tiny muscles (and nerves) involved in different movements of the face in the event that the target is not correctly determined, it is be easy inadvertently to cause unwanted weakening or paralysis of a facial movement.

According to one aspect of the invention there is provided a method of non-invasive treatment of the human body by ablating a muscle or a nerve controlling the muscle, the method comprising stimulating a muscle to emit an electromyographic signal, detecting the electromyographic signal to ascertain the location of the said muscle or alternatively the nerve, heating the said muscle or nerve by means of optical energy cooling the skin in the vicinity of the said muscle or nerve, and applying radio frequency energy to the muscle or nerve to ablate or partially to ablate said muscle or nerve.

According to another aspect of the invention there is provided a surgical appliance for ablating selected muscles or nerves of a patient comprising: an implement having a working surface which can be applied to the surface of the body of the patient, the said working surface carrying: a cathode and an anode with suitable electric powering devices, the cathode being arranged to emit an electric power pulse which will stimulate the targeted muscle; a detecting device which will detect an electromyographic (EMG) pulse emitted by the targeted muscle and which will emit a signal when it has so detected the said electric pulse so that the location of the target will be determined; a pair of spaced optical energy emitting devices which can heat the targeted muscle or nerve, cooling means arranged to cool the skin through which the optical energy must pass, and a pair of radio frequency electrodes connectable to a source of radio frequency electric current and being located so that when actuated they heat the target to oblate it.

The radio frequency electrodes are preferably bi-polar electrodes. They may be arranged to be connected selectively to the source of radio frequency electric current and to the electric powering device. The electrodes are located at a distance allowing a suitable depth of penetration conveniently being placed 12 to 16 mm apart to allow for a RF depth of penetration of approximately six to eight millimetres.

The optical energy emitting devices are preferably such as to provide an optical light wavelength long enough to reach the target organ with the energy levels of light are sufficiently great to preheat the organ so that the radio frequency can ablate or significantly impede target nerve or muscle function.

The optical energy emitting devices may comprise a laser conveniently having a sapphire light emitter.

An embodiment of the invention will now be described by way of example with reference to the accompanying drawing which shows a unit for use in cosmetic aesthetic treatment of the face.

Referring now to the drawing there is shown a surgical unit 10 of the invention. The unit 10 comprises a cylindrical operating implement 12 which is of such dimensions as to be able to be easily and accurately manipulated by a surgeon for example being in the shape of a thick pen having dimensions of 25 mm diameter and 210 mm long. The unit 10 further comprises two electric power packs 14 and 16 which provide radio frequency power to stimulate a muscle respectively. In addition there is a source 18 of cool gases. The power packs 14 and 16 which are connected to the rear end wall 20 of the implement 12 through suitable cabling 22 and 24. The source of cool gases 18 is connected thereto by a flexible fluid conduit 26. The cabling 22 is also connected to a detector box 28 to be described more fully below.

The front end of the implement 12 is comprised by a smooth and flat end working surface 30 lying at right angles to the axis 32 of the implement 12 and being connected to the cylindrical wall of the implement 12 by a rounded peripheral corner 32.

Formed on the end surface 30 are a pair of bi-polar electrodes 34 and 36. The electrodes 34 and 36 are spaced apart (centre to centre) by a distance equal to about twice the depth of the targeted muscle. Conveniently this distance is between 12 mm and 16 mm. By means of suitable switch means (not shown) the electrodes 34 and 36 can be connected to either power pack 14 and 16. Also formed on the end surface between the electrodes 34 and 36 is a rectangular sapphire light guide 38 forming part of a laser which is capable of emitting light in the range of 570 to 590 nm at an optical energy of 10 J/cm².

Within the unit 10 and having an end section running adjacent to the end surface 30 is an internal conduit 40 through which cool gases from the source 18 are directed.

Also within the implement 12 at the end surface 30 there is a preamplifier pod (not shown). This pod is arranged to carry out the following two actions. First it supplies power to the electrodes 32 and 334. Second it detects any EMG signal (i.e. any signal emitted by the muscle when stimulated) that is received by the electrodes 34 and 36 and transmits such EMG signal to the detector box 28.

The detector box 28 serves as a nerve monitor. It, the detector box 28, is connected via the cabling 22 to the bi polar electrodes 34 and 36 from which it receives an EMG signal. The detector box 28 has a display 42 for indicating a signal which has been received from a muscle as will be described. The display 42 may comprise an oscilloscope display unit but preferably may be a bar code display. The display preferably includes a control to change the colour of the display when the EMG signal increases to a level which would indicate that the location of the nerve or muscle. The detector box 28 may also incorporate an audible signal means which will provide an audible signal when the EMG signal increases to a level which would indicate that the location of the muscle has been located. Alternatively the audible signal means may merely amplify the EMG signal so that the surgeon will be able to ascertain therefrom the location of the muscle.

In use, the patient will be anaethtised normally by the surgeon will manipulate the implement to pass the end surface 30 of the implement 12 over the skin of a patient in the general location of the muscle which it is desired to ablate.

Direct current provided by the power pack 16 is applied to the bipolar electrodes to stimulate the muscles below the skin. The electrodes provide a 200μ pulse at an output range of 0.05 mA to 5 mA. When the implement is close enough to the muscle and/or the nerve controlling for the muscle to be bridged by the electrodes 34 and 36, the muscle will contract when it is acted upon by the pulse from the electrodes. During such contraction the EMG signals of very small voltage (of the order of a millionth to a few thousandth of a volt) are emitted. These signals are detected by the preamplifier pod which sends an appropriate signal to the detector box 28. The surgeon will now be aware of the location of the muscle that is to be ablated. The surgeon will now alter the setting of the implement so that the following actions take place. First the end surface 30 of the implement 12 is cooled by the coolant gases which pass through the internal conduit 40 to reduce the temperature of the surface 30 to about 10° C. which in turn will cool the dermis to about 10° C. The optical energy is now applied by the laser and this will raise the temperature of the targeted muscle relative to the surrounding tissue. The electrodes 34 and 36 are then connected by the pod to the radio frequency power pack 14 so that a radio frequency pulse is applied to the heated targeted muscle to ablate the muscle.

Because the distance apart of the electrodes 34 and 36 is between twelve and sixteen millimetres this will allow an RF depth penetration of approximately six to eight millimetres.

The degree of ablation is controlled by the surgeon who controlls the dosage of radio frequency applied. Desirably the ablation is not complete or irreversible. This will mean that the muscle will not cause the skin to appear to be paralysed but will only allow small and aesthetically acceptable movements under control of the muscle. Furthermore the muscle will in due course recover its strength. The dosage for ablation is preferably arranged so that muscle will regain its strength between three months and two years but preferably in about one year.

The treatment above described can be used inter alia on the corrugator muscles, procerus, lateral orbicularis levator alaeque nasi muscle, depressor anguli oris and platysma muscles, crows feet, glabella, brow depression, lower lid puffiness turnup tip of the nose, dimpling of the chine, smokers lines around the mouth and neck bands. Due to such ablation the muscles will not form corrugations or wrinkles on the skin with a significant aesthetic improvement of the face. In addition the treatment can be used to ablate the protagonist and antagonist muscles controlling turning down of the corners of the mouth and brow depression.

It has been found that with partial ablation of the muscle this will result in a softening of expression rather than an unnatural paralysis. After a period of time, depending upon the treatment, as menitoned above, the ablated muscle will regain its strength and further treatment will be required. This is generally regarded as a minor inconvenience when compared to the alternative of having a “paralysed look”.

It will be seen that by having the end surface 30 of the implement 12 on the surface of the skin of the patient, the EMG signal can more easily be detected. Furthermore this will avoid extraneous noise from affecting such detection.

It will be understood that the patient will normally be anaesthetised during the treatment. This is effected by a local anaesthetic block by blocking the nerve being ablated or that which controls muscle to be ablated. Such block taking place away from the actual area being treated. Typically when muscles in the upper lip are being ablated, an injection is given into the cheek just below the eye. Thus it can be seen that the patient will be awake during the procedure and the procedure can take place in the surgeon's rooms. Of course if desired or if the patient is having another procedure, the procedure of the invention can take place with the patient being under a general anaesthetic.

It will further be seen that this technique will provide very satisfactory non-invasive cosmetic and aesthetic treatment with the added advantage that no toxins are introduced into the body of the patient. The techniques provides the ability to act transcutaneously non-invasively (i.e without cutting the skin). All other treatments require cutting open the skin or piercing it with needles before delivering the RF dose. In addition the muscle (or nerve) is accurately targeted so that other tissues of the patient are not damaged during this process. This is an important advantage because there are so many tiny muscles (and nerves) involved in different movements of the face and it would be easy to cause unwanted weakening or paralysis of a facial movement if the target is incorrectly selected. It will also be noted that by stimulating the area before the RF shot, one is mimicking the movement that will be directly affected by the RF so the result becomes more predictable.

The invention is not limited to the precise details hereinbefore described. For example the surgeon may use the EMG signal to detect the location of the nerve which controls the particular muscle that is causing the unwanted wrinkling etc. By ablation of the nerve the action of the muscle will be reduced. The shape and form of the surgical device may vary as desired. The distance apart of the electrodes may vary in dependence upon the depth of the nerve or muscle which it is desired to locate and to ablate. As an alternative this may be that the electrodes located a about six to ten millimetres apart so that they can heat tissue between three and five millimetres below the dermis. If desired the electrodes can be mounted in a way which would permit their relative location to be adjusted. The surgeon may detect the position of the muscle to be ablated by seeing the movement of the muscle whifh is transmitted through the skin. Thus the detector box and display may be omitted.

1. A surgical apparatus for ablating a target area consisting of selected muscles or nerves of a patient, the surgical appliance comprising an implement having a working surface which can be applied to the surface of the body of the patient proximate to the target area, said working surface comprising:
a cathode and an anode with suitable electric powering devices, the cathode being arranged to emit an electric power pulse which will stimulate the target area;
a detecting device which will detect an electromyographic (EMG) pulse emitted by the target area and emit a signal in response to said detection so that the location of the target can be determined;
an optical energy emitting device which can heat the target area;
a cooling means arranged to cool the surface of the body of the patient proximate to the target area through which the optical energy must pass, and
a pair of radio frequency electrodes connectable to a source of radio frequency electric current and being located so that when actuated, the pair of radio frequency electrodes heat the target area to oblate it.

2. The apparatus of claim 1, wherein the pair of radio frequency electrodes are bi-polar electrodes.

3. The apparatus of claim 2, wherein the bi-polar electrodes are arranged to be connected selectively to the source of radio frequency electric current and to the electric powering device so that when connected to the powering device, they serve respectively as the said cathode and said anode.

4. The apparatus of claim 1, wherein the electrodes are located at a distance allowing a suitable depth of penetration of the radio frequency signal.

5. The apparatus of claim 4, wherein the electrodes are placed 12 to 16 mm apart to allow for an RF depth of penetration of approximately six to eight millimeters.

6. The apparatus of claim 1, wherein the optical energy emitting device operates to provide an optical light wavelength suffient to reach the target area with sufficient energy levels of light that preheat the target area so that the radio frequency can ablate or significantly heat muscle or nerve tissue within the target area.

7. The apparatus of claim 6, wherein the optical energy emitting devices provide light in the range of 570 to 590 nm at an optical energy of 10 J/cm²

8. The apparatus of claim 1, wherein the optical energy emitting device comprises a laser.

9. The apparatus of claim 8, wherein the laser has a sapphire light emitter.

10. The apparatus of claim 1, wherein the detector device incorporates a signaling means to indicate to a surgeon when the electromyographic signal is at a maximum.

11. The apparatus of claim 10, wherein the signaling means is a visual display.

12. The apparatus of claim 11, wherein the visual display changes color when the electromyographic signal approaches a maximum.

13. A method of non-invasive treatment of the human body by ablating a target consisting of a muscle or a nerve controlling the muscle, the method comprising the steps of:
stimulating a target to emit an electromyographic signal;
detecting the electromyographic signal to ascertain the location of the heating the target with optical energy;
cooling the skin in the vicinity of the target, and
applying radio frequency energy to the target to at least partially ablate said target.

14. The method of claim 12, wherein the target stimulated by means of a pulsed electrical stimuli of 2 Hz, 200 millisecond duration and 0.5 to 2.9 mA.

15. A cosmetic surgery method comprising the steps of:
stimulating a target area with an electronic pulse;
detecting a reaction to the electronic pulse;
using the reaction to identify the location of a tissue within the target area;
cooling surface tissue in the vicinity of the target area,
heating the target area; and
applying radio frequency energy to the tissue to at least partially ablate the tissue.

16. The method of claim 15, wherein the step of detecting a reaction to the electronic pulse comprises detecting an EMG signal.

17. The method of claim 16, wherein the step of detecting a reaction further comprises providing an indicator to signify the detection.

18. The method of claim 15, wherein the steps of cooling the surface tissue, heating the target area and applying radio frequency energy to the tissue are performed after the step of detecting a reaction.

19. The method of claim 18, wherein the step of heating the target area further comprises the step of applying optical energy that penetrates to the target area.

20. The method of claim 19, wherein the step of applying optical energy further comprises using a laser.
